(19) United States
(12) Patent Application Publication
(10) Pub. No.: US 2015/0045743 A1
(43) Pub. Date: Feb. 12, 2015

(54) MULTIPURPOSE SYRINGE ADAPTER

(71) Applicant: Gerald I. Fretwell, Elizabethtown, PA (US)

(72) Inventor: Gerald I. Fretwell, Elizabethtown, PA (US)

(21) Appl. No.: 13/959,961

(22) Filed: Aug. 6, 2013

(51) Int. Cl. A61M 5/34; A61M 39/20

(52) U.S. Cl. CPC A61M 5/345; A61M 39/20; A61M 2205/19
USPC 604/240

(57) ABSTRACT

A syringe adapter for use on multiple sized syringes comprising: a neck, where the neck is capable of connecting to a syringe; a flange positioned just below the neck; a base portion positioned abutting the flange positioned just below the flange; and a tapered sleeve below the flange. The syringe adaptor may further include a cap that inserts into the neck of the syringe adaptor during non-use. In one particular embodiment, the cap includes a top flat portion and a stem that goes into the neck. Also, the syringe adaptor may include a cover that fits over the tapered sleeve during non-use.

### BACKGROUND OF THE INVENTION

1. Field of Invention

The present invention relates to an adapter that is used on multiple syringes to eliminate the need to use one specific syringe and allows greater flexibility for the use of a single syringe.

2. Description of Related Art

In the medical field syringes are used to administer medication or to draw fluids for an individual. The typical syringe includes a pump consisting of a plunger that fits tightly in a tube. The cylindrical tube of the syringe is usually referred to as a barrel and allows the syringe to take in or expel liquid through an opening at the end of the tube. One aspect of syringes is the tip design of the syringe. The tip allows for the placement of a hypodermic needle. The most well-known tip design is the Luer Lok®. The Luer Lok® design allows for a system of fittings that include a male tapered fitting and a mating female part on medical and laboratory instruments such as the hypodermic needle. The sizes of the syringes vary for placement on different sized tubing to provide a connection for the insertion or removal of fluids. Many times during an emergency situation certain size syringes may not be available or there may be time lost in searching for the correct syringe size. Consequently, it would be advantageous to have an adaptor that could be placed upon any syringe and allow for the flexibility in use of a single syringe on multiple tubing in a medical environment.

### SUMMARY OF THE INVENTION

The present invention relates to a syringe adapter for use on multiple sized syringes comprising: a neck, where the neck is capable of connecting to a syringe; a flange positioned just below the neck; a base portion positioned abutting the flange positioned just below the flange; and a tapered sleeve below the flange. The syringe adaptor may further include a cap that inserts into the neck of the syringe adaptor during non-use. In one particular embodiment, the cap includes a top flat portion and a stem that goes into the neck. Also, the syringe adaptor may include a cover that fits over the tapered sleeve during non-use.

### BRIEF DESCRIPTION OF DRAWINGS

FIG. 1 depicts a multi-purpose syringe adaptor in accordance with the present invention.

### DETAILED DESCRIPTION

The present invention relates to a syringe adaptor that's placed upon a standard Luerlok syringe to provide an adaptor that can be used on various sized tubing during medical procedures. The present invention includes a adaptor that has a neck that fits onto the Luerlok of a syringe. The adaptor includes a cap and a cover that assures the adaptor remains sanitary prior to use. During use the cap is removed and sleeve removed at a tapered end of the adaptor. The neck of the adaptor is placed into the syringe and the tapered sleeve may be inserted into tubing regardless of size. This eliminates the need for having multiple sized syringes for different types tubing in a medical environment.

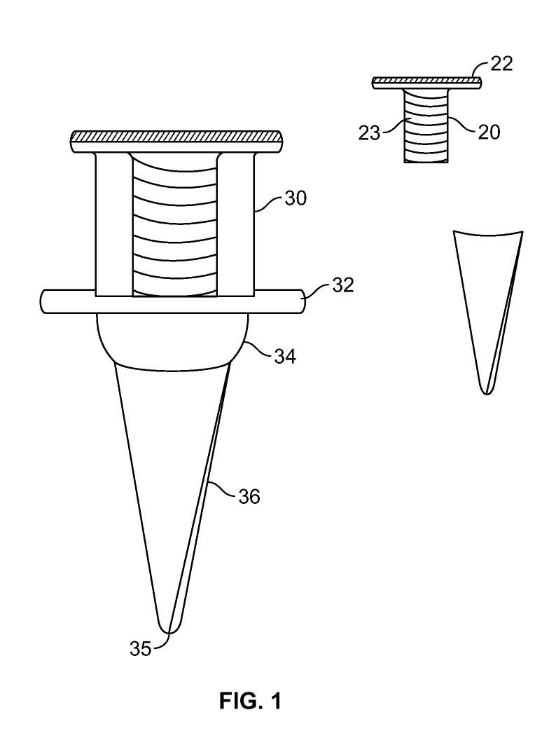

In reference to FIG. 1, a syringe adaptor in accordance with the present invention is depicted. The syringe adaptor includes a fitting neck 30 that extends above a flange 32. Below the flange 32 is a base portion 34. Below the base portion 34 is a tapered sleeve 35 that allows for the insertion of the adaptor into various sized tubing to fit onto the sleeve 35. Over the sleeve 35 is a cover 36 that remains in place until the adaptor is ready to be used by a medical professional. A cap 20 remains within the neck 30 prior to insertion onto a syringe. The cap 20 and cover 36 was shown separately to provide a clear view of these components. The cap 20 includes a top flat portion 22 and a stem 23 that goes into the neck 30 of the adaptor.

During use this adaptor may be used upon any syringe that includes the standard Luer Lok® connection system that is widely used throughout the medical profession. The adaptor's neck 30 is inserted upon the Luer Lok® and then the cover 36 is removed from the sleeve 35 for insertion into tubing. Use of the present invention eliminates the need for using multiple syringes and allows a professional to use one specific syringe to administer meds or to administer flushes as needed. The instant invention has been shown and described in what it considers to be the most practical and preferred embodiments. It is recognized, however, that departures may be made there from within the scope of the invention and that obvious modifications will occur to a person skilled in the art.

What is claimed is:

1. A syringe adapter for use on multiple sized syringes comprising:
a. a neck, where the neck is capable of connecting to a syringe;
b. a flange positioned just below the neck;
c. a base portion positioned abutting the flange positioned just below the flange; and
d. a tapered sleeve below the flange.

2. The syringe adaptor according to claim 1, further including a cap that inserts into the neck of the syringe adaptor during non-use.

3. The syringe adaptor according to claim 2, where the cap includes a top flat portion and a stem that goes into the neck.

4. The syringe adaptor according to claim 1, further including a cover that fits over the tapered sleeve during non-use.
